(19) United States
(12) Patent Application Publication
(10) Pub. No.: US 2014/0078469 A1
(43) Pub. Date: Mar. 20, 2014

(54) SLIT LAMP UNIT FOR A SURGICAL MICROSCOPE

(71) Applicant: Leica Microsystems (Schweiz) AG, Heerbrugg (CH)

(72) Inventor: Michael GUENTERT, Heerbrugg (CH)

(73) Assignee: Leica Microsystems (Schweiz) AG, Heerbrugg (CH)

(21) Appl. No.: 14/024,671

(22) Filed: Sep. 12, 2013

(30) Foreign Application Priority Data
Sep. 14, 2012 (DE) 10 2012 216 470.7

(51) Int. Cl. A61B 3/135

(52) U.S. Cl. CPC A61B 3/135
USPC 351/214

(57) ABSTRACT

The present invention relates to a slit lamp unit (1) for a surgical microscope, the slit lamp unit (1) comprising a slit illumination unit (2) having a slit illumination optic for generating a slit illumination beam path (5, 6), and a guide rail (3) for displacement of the slit illumination unit (2) along a direction designated by the guide rail (3), the guide rail being embodied for displacement of the slit illumination unit (2) along a linear direction (12), and the slit illumination unit (2) being mounted on the guide rail (3) rotatably around a rotation axis (13) perpendicular to said linear displacement direction (12).

### CROSS REFERENCE TO RELATED APPLICATIONS

This application claims priority of German patent application number 10 10 2012 216 470.7 filed Sep. 14, 2012, the entire disclosure of which is incorporated by reference herein.

### FIELD OF THE INVENTION

The present invention relates to a slit lamp unit for a surgical microscope and to a surgical microscope having such a slit lamp unit, the slit lamp unit comprising a slit illumination unit having a slit illumination optic for generating a slit illumination beam path, and a guide rail for displacement of the slit illumination unit along a direction designated by the guide rail. Surgical microscopes having slit illumination are also referred to as “surgical slit lamps.”

### BACKGROUND OF THE INVENTION

Slit lamps used for ophthalmic investigations have been known for some time from the existing art. Surgical or diagnostic slit lamps are made up of the actual slit lamp unit, the microscope, which may be a stereomicroscope, and the mechanical device (adapter) for coupling the microscope and slit lamp unit.

The function of the slit lamp unit is to generate, on the object being investigated, a slit image that is as bright as possible. The Köhler illumination principle, in which the light source is imaged by a collector system into the objective, is usually used for this. The objective in turn images the slit, arranged in front of the light source, into the object plane. This type of illumination furnishes a very homogeneous slit image regardless of the structure of the light source.

A stereomicroscope is usually utilized as a microscope for a diagnostic or surgical slit lamp of this kind Reference may be made to the extensive existing art regarding the construction and mode of operation of stereomicroscopes or surgical microscopes. A mechanical device couples the slip lamp unit to the stereomicroscope. With diagnostic slit lamps, the slit image is directed by means of deflection prisms onto the object (human eye), while the stereomicroscope is arranged “downstream” from the slit lamp unit in such a way that the space located between the main objective of the microscope and the object (eye) can be occupied at least in part by the slit illumination unit of the slit lamp unit. The aforesaid mechanical device for coupling the microscope and slit lamp unit allows the slit illumination light beam path to be pivoted around the observation beam path within a specific angle, the pivot plane extending substantially through the stereo base of the stereomicroscope on the one hand and through the connecting line of the eyes on the other hand. Surgical slit lamps are a combination of a surgical microscope with a pivotable slit illumination system, in which context the slit illumination unit can be arranged below the main objective on the microscope body. A circular-arc-shaped guide rail permits adjustment of the slit illumination unit over a relatively large angle (40° to 60°), the center point of the relevant circle being located substantially in the object to be investigated in the object plane.

In a surgical slit lamp, the type of object illumination depends principally on whether the anterior or posterior ocular media are to be made visible. If the anterior ocular media are to be made visible, the slit is imaged so that the largest possible angle exists between the observation axis and illumination axis, a light sheet is placed into the anterior ocular segment, and the ocular background remains dark. Regions beyond the lens and as far as the retina of the eye, on the other hand, are often viewed in regredient light (red reflex). Here the object structure is observed by transmitted red light, and thus detected by way of differences in absorption. The red light itself is generated by direct reflection at the surfaces of the lens or at the fundus (retina). For this, the angle between the observation axis and illumination axis must be minimized.

DE 10 2009 026 455 A1 discloses a slit illumination unit having a deflection element for a slit illumination beam path, which element directs said beam path via the main objective of a stereomicroscope onto the object plane. According to this document, the deflection element for the slit illumination beam path is arranged on the axis of a stereo base of the stereomicroscope. The deflection element is consequently located substantially on a connecting line between the axes of the observation channels, and is arranged between the main objective and the tube lenses of the stereomicroscope. For further details in this regard and with regard to surgical slit lamps in general, reference may be made to this document.

DE 196 50 773 A1 discloses an illumination apparatus for a surgical microscope, in particular for ophthalmological procedures, with which apparatus a coaxial zero-degree illumination for achieving a red reflex, and a 6-degree illumination can be selected. The relative brightness levels of the coaxial zero-degree illumination and of the 6-degree illumination can be selected as desired by means of a suitable arrangement of deflection elements arranged above the main objective (as viewed from the object).

DE 42 14 445 A1 discloses an ophthalmic surgical microscope for achieving an optimum red reflex. Depending on the respective magnification of the surgical microscope and on the object being investigated, the width of a so-called “zero degree deflection element” in the direction of the connecting line of the two stereo observation beam paths can be varied here via suitable adjustment devices.

As already mentioned, in surgical slit lamps known hitherto the slit illumination unit is suspended on a circular-arc segment whose radius matches the working distance (WD) of the microscope. Different illumination angles can thus be established by displacing the illumination unit. If the working distance changes, however, in the context of an objective change and/or establishment of a different magnification (e.g. using a variable objective), the circular-arc element along with the entire illumination unit must be interchanged. Separate slit illumination units are thus necessary for each working distance (typically 175, 200, or 225 mm).

### SUMMARY OF THE INVENTION

The underlying object of the present invention is therefore to describe a slit lamp unit, and a surgical microscope having such a slit lamp unit, in which simple adaptation of the slit illumination to different working distances is possible.

This object is achieved by a slit lamp unit, and by a surgical microscope having such a slit lamp unit, in accordance with the features and preferred embodiments described herein.

The generic slit lamp unit is characterized according to the present invention in that a guide rail is embodied for displacement of the slit illumination unit along a linear direction; and that the slit illumination unit is mounted on the guide rail rotatably around a rotation axis perpendicular to said linear displacement direction in order to direct the slit illumination beam path from the selected position along the guide rail onto a desired location in the object plane of a surgical microscope. It is advantageous for this purpose if the slit illumination unit is rotatable around a rotation axis that is perpendicular to a plane that is spanned by the desired location in the object plane of the microscope and a straight line parallel to the linear displacement direction. Expressed in the coordinate system of the microscope, this means that the rotation axis is perpendicular to a plane that in turn is perpendicular to the object plane. The latter plane can, in particular, extend through the focus of the microscope and can contain the main axis of the microscope objective.

The slit lamp unit according to the present invention makes it possible to set up the slit illumination for different working distances with no need for major mechanical modification work, for example replacement of a circular-arc-shaped guide rail or the like. For adaptation to different working distances, the slit illumination unit merely needs to be displaced in a linear direction along the guide rail, and then suitably oriented by rotation around its rotation axis, so that the slit is imaged at the desired point in the object space. The slit illumination unit of a slit lamp unit according to the present invention thus exhibits a translational degree of freedom (x) and a rotational degree of freedom (β). Adjustments in accordance with these degrees of freedom can be performed under manual or preferably motorized control.

While a limitation to a specific working distance (WD), of e.g. 200 mm, hitherto existed when working with a slit lamp unit, different working distances can be implemented with the slit lamp unit according to the present invention. As a rule, a change in the working distance also requires a focus change in the focused slit image, so that it is advantageous if the slit illumination optic comprises a focusing optic that focuses the slit illumination beam path onto the respectively desired focal point.

It is advantageous if a respective motorized drive system for displacement of the slit illumination unit along the guide rail and/or for rotation of the slit illumination unit around the rotation axis is present. The two motions, i.e. translation and rotation, can be coupled to one another for a specific working distance (WD). In addition, the rotary motion and the adaptation of the focus by way of the focusing optic can be controlled in directly motorized fashion with the aid of a control unit. In this manner, a control unit can control, for a specific working distance, both the rotary motion and the focus adaptation as a function of location (x) in the linear direction of the guide rail. Control of this kind can be implemented for different working distances. Different working distances are necessitated by an objective change or by a change in magnification at a variable objective.

The invention furthermore relates to a surgical microscope having a slit lamp unit according to the present invention as described above. Surgical microscopes are sufficiently known from the existing art. The linear displacement direction of the slit illumination unit extends, in particular, parallel to the object plane of the microscope.

The slit illumination optic preferably encompasses a focusing optic that is connected to a first control unit in order to focus the slit illumination beam path onto the object plane of the microscope as a function of a working distance at the microscope and/or as a function of a linear displacement of the slit illumination unit.

In addition, the slit illumination unit is advantageously connected to a second control unit in order to direct the slit illumination beam path toward the focus of the microscope by corresponding rotation of the slit illumination unit around its rotation axis. Instead of the focus of the microscope, it is also possible to designate any other point in the object plane onto which the focused slit image is to arrive.

Advantageously, the aforesaid first and second control units are implemented together in a single control device.

Further advantages and embodiments of the invention are evident from the description and the appended drawings.

It is understood that the features recited above and those yet to be explained below are usable not only in the respective combination indicated, but also in other combinations or in isolation, without departing from the scope of the present invention.

The invention is schematically depicted in the drawings on the basis of an exemplifying embodiment and will be described in detail below with reference to the drawings.

### BRIEF DESCRIPTION OF THE DRAWING VIEWS

FIG. 1 is a schematic front view of an embodiment of a slit lamp unit according to the present invention;

FIG. 2 is a schematic side view of the slit lamp unit of FIG. 1 with the slit illumination unit having its slit illumination optic; and

FIG. 3 shows an embodiment of a surgical microscope having a slit lamp unit, in an embodiment according to the present invention.

### DETAILED DESCRIPTION OF THE INVENTION

The Figures are discussed below continuously. Identical reference characters refer to identical elements.

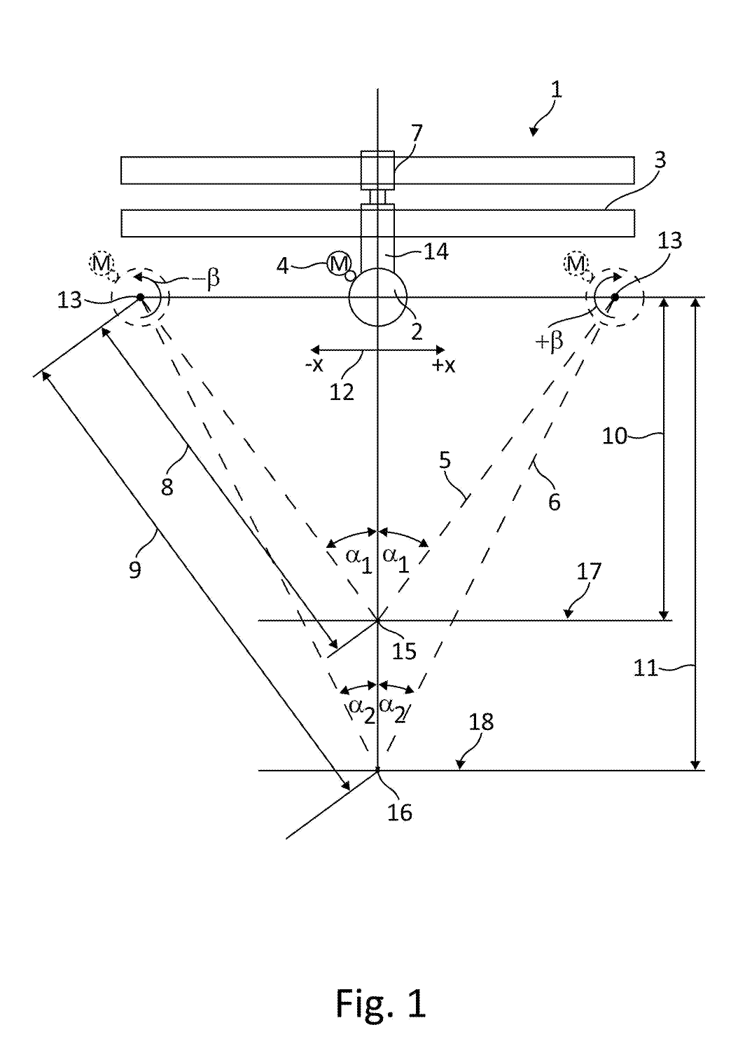

FIG. 1 very schematically shows an embodiment of a slit lamp unit 1 according to the invention for a surgical microscope. Slit lamp unit 1 encompasses a slit illumination unit 2 having a slit illumination optic for generating a slit illumination beam path, two possible slit illumination beam paths 5 and 6 being depicted here. The slit lamp unit further encompasses a guide rail 3 for displacement of slit illumination unit 2 along a linear X direction 12. Slit illumination unit 2 is correspondingly connected for this purpose to guide rail 3 via a suspension system 14. Slit illumination unit 2 is furthermore mounted on guide rail 3 rotatably around a rotation axis 13. The rotation angle is labeled β. Two symmetrical X displacement positions for illumination unit 2, as well as two likewise symmetrical rotation angles β of illumination unit 2 around a rotation axis 13, are drawn in FIG. 1. At least one encoder 7 measures the linear X displacement and/or rotation angle β of the illumination unit.

It is evident from FIG. 1 that axis 13 is perpendicular to a plane that is spanned by focal point 15 (or 16) of a slit illumination beam path 5 (or 6) and a straight line parallel to linear displacement direction 12. In FIG. 1, this is the drawing plane. This plane in turn is perpendicular to object plane 17 (or 18).

A motorized drive system for displacement of slit illumination unit 2 along guide rail 3 is labeled 4. A further drive system can control in motorized fashion the rotation of illumination unit 2 around rotation axis 13. The two drive systems can also be coupled to one another, since for a fixed working distance 10 (or 11), the magnitude of the X displacement is correlated with the magnitude of angle β. The relevant working distance 10 (or 11) can then be inputted into a control unit that thereupon adjusts angle β as a function of the X displacement.

To make it possible to work with different working distances 10 (or 11), it is useful if slit illumination unit 2 possesses a focusing optic that focuses the slit illumination beam path onto object plane 17 (or 18) as a function of a working distance 10 (or 11) and/or as a function of a linear displacement of slit illumination unit 2. The corresponding focal lengths are respectively labeled 8 and 9. The focal lengths are determined uniquely from the respective working distance and from the X position of illumination unit 2. The focal length can in turn be set or established by way of a control unit. Advantageously, all the control units are implemented in one control device.

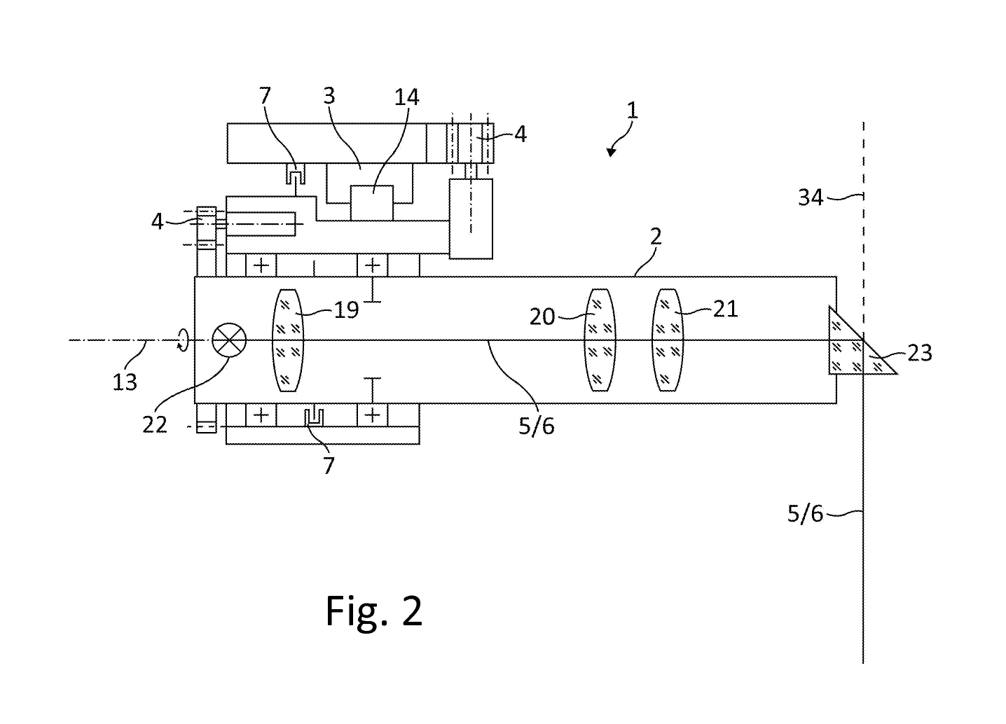

FIG. 2 is a side view, in section, of one possible embodiment of a slit illumination unit 2 of FIG. 1. Evident therefrom firstly is the slit illumination optic with its light source 22 and its lenses 19, 20, and 21. A focusing optic can be realized by displacement of at least one of these lenses. Further optical elements, such as apertures or the like, can be present as constituents of the slit illumination optic without being discussed here individually. Motorized drive systems 4 serve on the one hand to displace slit illumination unit 2 along guide rail 3 and on the other hand to rotate illumination unit 2 around rotation axis 13. The linear X displacement resulting therefrom, as well as rotation angle β, can be measured and determined by means of encoders 7. For a predetermined working distance, rotation angle β can be set or established by means of a control unit (not depicted) as a function of the linear X displacement. The focal length can then be readjusted accordingly by means of another control unit or a higher-order control device.

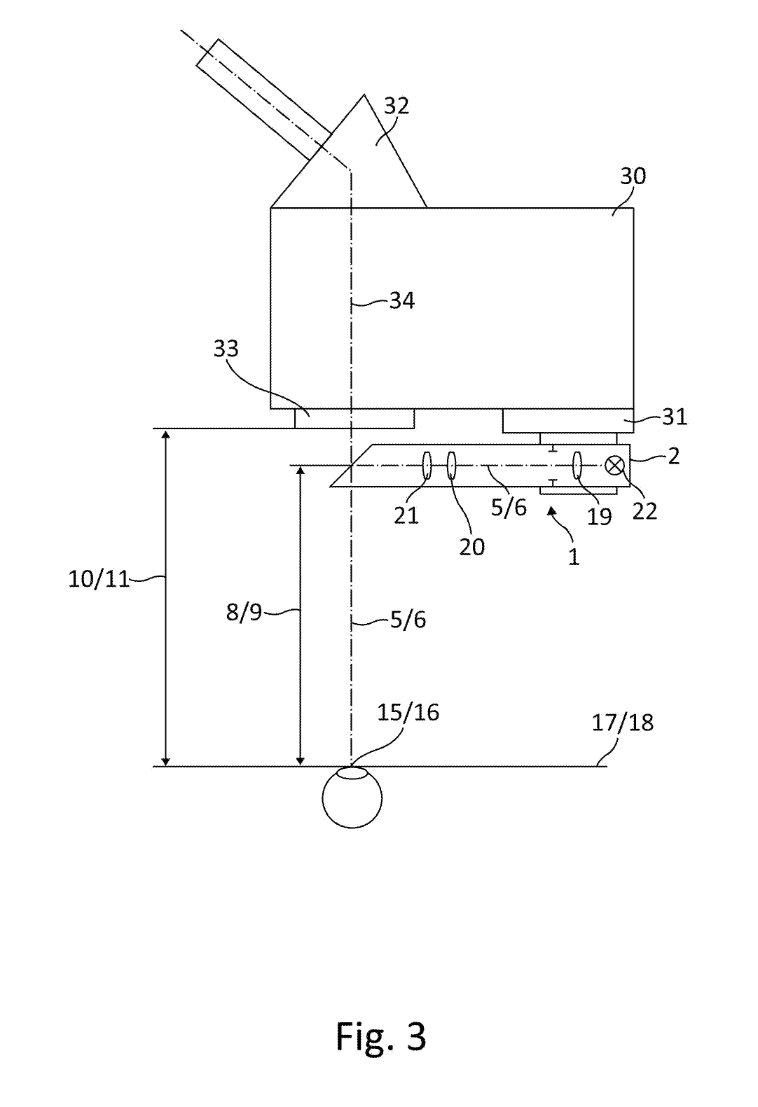

Lastly, FIG. 3 shows a surgical microscope 30 having a slit lamp unit 1 (also called a surgical slit lamp). The mechanical device, i.e. adapter, for coupling microscope 30 and slit lamp unit 1 is labeled 31. Surgical microscope 30 encompasses a tube 32 and a main objective 33. The construction and mode of operation of a surgical microscope will not be discussed in detail below. It is apparent from FIG. 3 that adapter 31 mechanically couples surgical microscope 30 to slit lamp unit 1. It is further apparent that deflection prism 23 (see FIG. 2) directs first or second slit illumination beam path 5, 6 respectively onto first or second object plane 17, 18. The focusing optic of the slit illumination optic correspondingly focuses the slit image respectively onto first or second focal point 15, 16. A light sheet can thereby be placed into the anterior ocular segment. The associated focal lengths are respectively labeled 8 and 9, and the associated working distances respectively 10 and 11. For details of this, reference is made to FIG. 1 and the explanations thereof. An observer at surgical microscope 30 can thereby obtain a magnified image of the anterior ocular media. The associated observation beam path is labeled 34.

### PARTS LIST

1 Slit lamp unit

2 Slit illumination unit

3 Guide rail

4 Motorized drive system

5 First slit illumination beam path

6 Second slit illumination beam path

7 Encoder

8 First focal length

9 Second focal length

10 First working distance

11 Second working distance

12 Linear displacement direction

13 Rotation axis

14 Suspension system

15 First focal point

16 Second focal point

17 First object plane

18 Second object plane

19 Lens of slit illumination optic

20 Lens of slit illumination optic

21 Lens of slit illumination optic

22 Light source of slit illumination optic

23 Deflection prism

30 Surgical microscope

31 Adapter

32 Tube

33 Main objective

34 Observation beam path

What is claimed is:

1. A slit lamp unit (1) for a surgical microscope (30), the slit lamp unit (1) comprising:
a slit illumination unit (2) having a slit illumination optic (22, 19, 20, 21) for generating a slit illumination beam path (5, 6); and
a guide rail (3) for guiding displacement of the slit illumination unit (2) along a direction designated by the guide rail, wherein the direction is a linear direction (12);
wherein the slit illumination unit (2) is mounted on the guide rail (3) rotatably around a rotation axis (13) extending perpendicular to the linear displacement direction (12).

2. The slit lamp unit according to claim 1, wherein the slit illumination optic (22, 19, 20, 21) includes a focusing optic that focuses the slit illumination beam path (5, 6) onto a desired focal point (15, 16).

3. The slit lamp unit according to claim 2, wherein the rotation axis (13) is perpendicular to a plane spanned by the focal point (15, 16) of the slit illumination beam path (5, 6) and a straight line parallel to the linear displacement direction (12).

4. The slit lamp unit according to claim 1, further comprising a motorized drive system (4) for displacement of the slit illumination unit (2) along the guide rail (3).

5. The slit lamp unit according to claim 1, further comprising a motorized drive system (4) for rotation of the slit illumination unit (2) around the rotation axis (13).

6. The slit lamp unit according to claim 4, wherein the motorized drive system (4) is operable to rotate the slit illumination unit (2) around the rotation axis (13).

7. A surgical microscope (30) comprising:
a microscope objective (33)
a slit lamp unit (1), the slit lamp unit (1) comprising a slit illumination unit (2) having a slit illumination optic (22, 19, 20, 21) for generating a slit illumination beam path (5, 6); and
a guide rail (3) for displacement of the slit illumination unit (2) along a direction designated by the guide rail (3), wherein the direction is a linear direction (12) parallel to an object plane (17, 18) of the microscope (30);
wherein the slit illumination unit (2) is mounted on the guide rail (3) rotatably around a rotation axis (13) extending perpendicular to the linear displacement direction (12).

8. The surgical microscope according to claim 7, wherein the rotation axis (13) is perpendicular to a plane that in turn is perpendicular to the object plane (17, 18).

9. The surgical microscope according to claim 7, wherein the slit illumination optic (22, 19, 20, 21) includes a focusing optic, and wherein the surgical microscope further comprises a first control unit connected to the focusing optic, the first control unit being operable to focus the slit illumination beam path onto the object plane (17, 18) of the microscope.

10. The surgical microscope according to claim 9, wherein the first control unit focuses the slit illumination beam path as a function of a working distance (10, 11) at the microscope.

11. The surgical microscope according to claim 9, wherein the first control unit focuses the slit illumination beam path as a function of a linear displacement (x) of the slit illumination unit (2).

12. The surgical microscope according to claim 9, wherein the first control unit focuses the slit illumination beam path as a function of a working distance (10, 11) at the microscope and a linear displacement (x) of the slit illumination unit (2).

13. The surgical microscope according to claim 9, further comprising a second control unit for controlling rotation (β) of the slit illumination unit (2) around the rotation axis (13) to direct the slit illumination beam path (5, 6) toward a focal point (15, 16) of the microscope.

14. The surgical microscope according to claim 13, wherein the first control unit and the second control unit are implemented together in one control device.

15. The surgical microscope according to claim 7, wherein the slit lamp unit (1) has a motorized drive system (4) for displacement of the slit illumination unit (2) along the guide rail (3).

16. The surgical microscope according to claim 7, wherein the slit lamp unit (1) has a motorized drive system (4) for rotation of the slit illumination unit (2) around the rotation axis (13).

17. The surgical microscope according to claim 15, wherein the motorized drive system (4) is operable to rotate the slit illumination unit (2) around the rotation axis (13).
